(19) United States
(12) Patent Application Publication
(10) Pub. No.: US 2010/0249857 A1
(43) Pub. Date: Sep. 30, 2010

(54) SURGICAL APPARATUS

(76) Inventors: Gursharan Singh Chana, West Midlands (GB); Christopher John Sloss, South Yorkshire (GB)

Correspondence Address: PATENTTM.US, P. O. BOX 82788, PORTLAND, OR, 97282-0788, US

(21) Appl. No.: 12/438,001

(22) PCT Filed: Sep. 4, 2007
(86) PCT No.: PCT/GB2007/003311

§ 371 Date: Jun. 15, 2010

(30) Foreign Application Priority Data
Sep. 5, 2006 (GB) 0617484.1
Apr. 27, 2007 (GB) 0708169.8

(51) Int. Cl. A61F 2/46

(52) U.S. Cl. 606/86 R

(57) ABSTRACT

A surgical apparatus (10) comprises a manipulating portion (12), a delivery portion (14) spaced longitudinally from the manipulating portion, and a connecting portion (16) connecting the manipulating portion to the delivery portion. The connecting portion extends laterally from the delivery portion. The surgical apparatus further includes a securing arrangement (28) for releasably securing a surgical member to the delivery portion.

This invention relates to surgical apparatus. More particularly, but not exclusively, this invention relates to surgical apparatus for use in implanting surgical members during operations, for example hip replacement operations. The surgical members may comprise cup implants for implantation into the acetabulum of a patient.

During surgery, such as hip replacement surgery, it is desirable to cause minimal damage. Known surgical apparatus for implanting cups into the acetabulum require large incisions, in order to be used. However, such surgical apparatus is difficult to use in minimal invasive hip replacement surgery.

According to one aspect of this invention, there is provided a surgical apparatus comprising a manipulating portion, a delivery portion spaced longitudinally from the manipulating portion, and a connecting portion connecting the manipulating portion to the delivery portion, wherein the connecting portion extends laterally from the delivery portion, and the surgical apparatus further includes a securing arrangement for releasably securing a surgical member to the delivery portion.

The surgical member may comprise a surgical implant, such as a cup implant for use in hip replacement surgery.

The connecting portion may be curved. The connecting portion may comprise a laterally extending region extending laterally from the delivery portion and may comprise a curved region which curves from the laterally extending region to the manipulating portion.

The surgical apparatus may comprise a manipulating device, which may comprise the manipulating portion, the delivery portion, and the connecting portion. The manipulating device may be generally question mark shaped. The surgical apparatus may further include a holding device on which the surgical member is mounted.

The holding device may be releasably securably mounted on the manipulating device. Conveniently, the holding device is releasably securably mounted on the delivery portion. The holding device may comprise a locking assembly for releasably locking the surgical member to the holding device.

The securing arrangement may comprise a securing mechanism operable to secure the holding device to the delivery portion. The securing mechanism may co-operate with the locking assembly to lock the surgical member to the holding device. The securing mechanism may co-operate with the locking assembly to release the surgical member from the holding device.

The securing mechanism may comprise a lever assembly, which may be pivotally mounted on the connecting portion. The lever assembly may be movable between a release position, in which the holding device can be arranged on or removed from the delivery portion, and a locking position, in which the holding device can be locked to the delivery portion, and in which locking position, the lever assembly can co-operate with the securing mechanism to secure the surgical member to the holding device.

The lever assembly may be movable between the release and the locking positions in a main plane of movement of the lever assembly.

The lever assembly may be movable to an intermediate position, in which the fastening mechanism can release the surgical member from the holding device, and in which intermediate position, the holding device can be locked to the delivery portion. The intermediate position may be between the release position and the locking position.

The lever assembly may comprise an urging arrangement to urge the lever assembly to the intermediate position from the release position. Conveniently, the urging arrangement urges the lever assembly to the intermediate position from the locking position. The urging arrangement may comprise a spring.

The lever assembly may comprise a lever. The lever assembly may have a proximal end at, or adjacent to, the manipulating portion, and a distal end at, or adjacent to, the delivery portion.

In one embodiment, the urging arrangement may comprise first and second springs. Each spring may be arranged at a respective opposite end region of the lever. Each of the first and second springs may comprise a respective lever spring. The lever assembly may be formed as an integral one-piece unit, for example, by casting, forging, moulding or the like.

In one embodiment, one of the first and second springs may be arranged to urge the lever assembly from the release position to the intermediate position. The other of the first and second springs may be arranged to urge the lever assembly from the locking position to the intermediate position.

In another embodiment, both of the first and second springs may be arranged to urge the lever assembly from the release position to the intermediate position. In this embodiment, both of the first and second springs may be arranged to urge the lever assembly from the locking position to the intermediate position.

The first and second springs may each comprise a respective leaf spring. Each of the first and second springs may extend from the lever to engage the connecting portion. Each leaf spring may have a first end attached to the lever, and a second end in contact with the connecting portion.

A pivot member may be provided in the connecting portion, and the lever assembly may be pivotally mounted on the pivot member to allow pivoting of the lever assembly about the pivot member.

The first spring may be provided at the distal end region of the lever assembly. The second spring may be provided at the proximal end region of the lever assembly.

The pivot member may comprise a screw threadably received in the connecting portion. The pivot member may extend through an aperture in the lever assembly. The aperture may be defined in the lever.

The lever assembly may extend to the delivery portion and a projection member may be provided on the lever assembly to engage the holding device, thereby locking the holding device to the delivery portion when the lever assembly is in the locking or the intermediate position. The projection member may be provided at the distal end region of the lever assembly.

When the lever assembly is in the intermediate position, the projection member may project from the delivery portion. Thus, the projection member may be received by the holding device to secure the holding device on the delivery portion when the lever assembly is in the intermediate position.

The lever assembly may include a fastener to fasten the lever assembly in the locking position. The fastener may comprise a detent member, which may be co-operable with a corresponding detent element on the lever assembly. The fastener may be movable between a fastening position, in which the lever assembly is fastened in the locking position, and a non-fastening position, in which the lever assembly can be moved from the locking position to the intermediate position.

The fastener may include an urging element to urge the fastener to the locking position. The urging element may comprise a spring, such as a leaf spring. The fastener may be formed as a one-piece unit, such as by casting, forging, moulding, or the like. The fastener may be provided at the proximal end region of the lever assembly.

In a first embodiment, the fastener may be movable between the fastening and the non-fastening positions substantially in the main plane of movement of the lever assembly. In the first embodiment, the urging element may be held within the connecting portion.

In a second embodiment, the fastener may be movable between the fastening and the non-fastening positions transverse to the main plane of movement of the lever assembly. Desirably, the fastener is movable between the fastening and the non-fastening positions at right angles to the main plane of movement of the lever assembly.

In the second embodiment, the urging element may be provided externally of the connecting portion, and the detent member may extend through the connecting portion. The urging element may extend along the connecting portion.

The fastener may comprise a pin movable between the fastening and the non-fastening positions transverse to the main plane of movement of the lever assembly.

The pin may be movable between the fastening and the non-fastening positions at right angles to the main plane of movement of the lever assembly. The pin may comprise an engagement end region configured to engage the lever assembly, and the lever assembly may define a detent recess to receive said engagement end region to hold the lever assembly in the locking position.

The pin may include a narrow region adjacent the aforesaid end region. The lever assembly may be movable between its positions when the narrow region is aligned with the lever assembly.

The urging element may be provided externally of the connecting portion, and the detent member may extend through the connecting portion.

The holding device may comprise a support member configured to support the surgical member. Desirably, the support member has substantially the same profile as the surgical member. The aforesaid plan profile may be a circular, or a generally circular, profile.

The locking assembly may be provided on the support member. The locking assembly may be movable between a locked position to lock the surgical member to the holding device, and an unlocked position to allow the surgical member to be mounted on, or removed from, the holding device.

The locking assembly may comprise a locking member. The locking assembly may comprise a plurality of locking members.

Each locking member may extend radially outwardly from a central region. Where the locking assembly comprises a plurality of locking members, each locking member may be spaced circumferentially from the other locking members.

A spring arrangement may be provided to urge the, or each, locking member to the unlocked position. In one embodiment, where the locking assembly comprises a plurality of circumferentially arranged locking members, the spring arrangement comprises a plurality of spring elements arranged between adjacent locking members.

In this embodiment, each spring element is of an M shaped configuration, and may be connected at its opposite ends to adjacent locking members.

The support member may define a socket to receive the locking assembly. The socket may be configured to be generally the same shape as the locking assembly. The socket may guide the locking assembly between the locked and the unlocked positions.

The delivery portion may comprise a platform surface on which the holding device can be mounted. A securing formation may be provided on the platform surface. The securing formation may comprise a raised portion extending from the platform surface.

A flange member may extend from the raised portion. The flange member may extend generally parallel to the platform surface, and may define with the platform surface, a securing channel to co-operate with a corresponding formation on the holding device.

The holding device may be slidably mounted on the delivery portion, and the support member may define an elongate recess to receive the securing formation on the delivery portion. The recess may be defined by a wall and an inwardly directed shoulder configured to co-operate with the securing formation on the delivery portion. The inwardly directed shoulder may be configured to co-operate with the securing channel on the delivery portion.

The projection member of the delivery portion may be operable to move the locking assembly to the locked position. The holding device may include a cam member for translating movement from the projection member to the, or each, locking member. The projection member may push the cam member. The projection member may comprise a pin. The projection member may be removably mounted on a distal end of the lever assembly.

The cam member may be of a generally conical configuration, having a conical, or frustoconical, cam surface. The cam member may engage the, or each, locking member to move the, or each, locking member to the locked position.

Each locking member may comprise a main portion for engaging the surgical member, and a cam engaging portion for engaging the cam member.

The projection member may engage the cam member to move the cam member away from the delivery portion, whereby the cam surface engages the, or each, locking member to move the, or each, locking member outwardly.

The holding device may comprise a cover member to hold the locking assembly on the support member. The cover member may define a bore or aperture to guide the cam member. The cam member may include a generally cylindrical region received in the bore or aperture in the cover member.

The cover member may protect the surgical member from damage. The cover member may be formed of an acetal or similar material. Suitable attaching members may be provided to attach the cover member to the support member. The attaching members may comprise screws.

According to another aspect of this invention, there is provided a holding device comprising a support member and a locking assembly to secure a surgical member to the holding device, wherein the locking assembly is mounted on the support member and comprises a locking member and a spring arrangement, the locking member being movable between a locked position and an unlocked position, and the spring arrangement is configured to urge the locking member to the unlocked position.

The holding device of this aspect of the invention may comprise the features of the holding device described above.

The surgical member may comprise an implant, such as a cup implant. The implant may be suitable for a hip joint. The surgical member may comprise a socket to receive a corresponding ball. A raised circumferential ridge may extend around the socket and may define a recess region to receive the, or each, locking member, thereby allowing the surgical member to be releasably locked to the holding device.

According to another aspect of this invention, there is provided a surgical member, comprising a socket for receiving a ball, and a raised ridge extending around the socket, wherein the ridge defines at least one recess to region to receive a locking member of a holding device, to allow the surgical member to be releasably locked to the holding device.

The, or each, recess region may be in the form of a groove. The ridge may define a plurality of recess regions, which may be spaced around the ridge. The recess regions may be substantially evenly spaced around the ridge.

An embodiment of the invention will now be described by way of example only with reference to the accompanying drawings, in which:

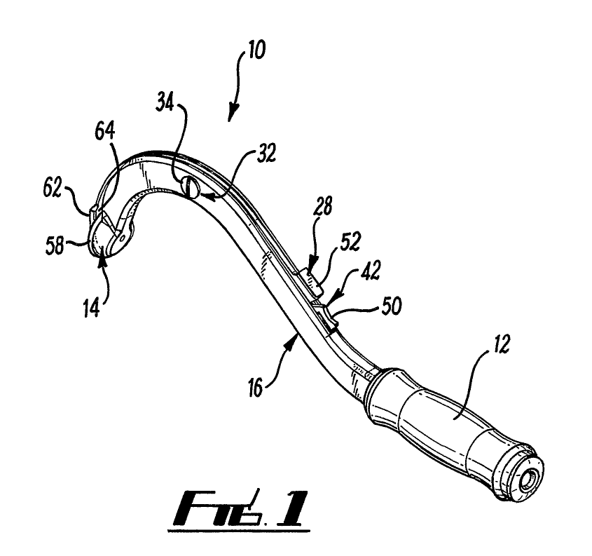

FIG. 1 is a perspective view of a first embodiment of a manipulating device of a surgical apparatus;

FIG. 2 is a part sectional side view of the manipulating device shown in FIG. 1;

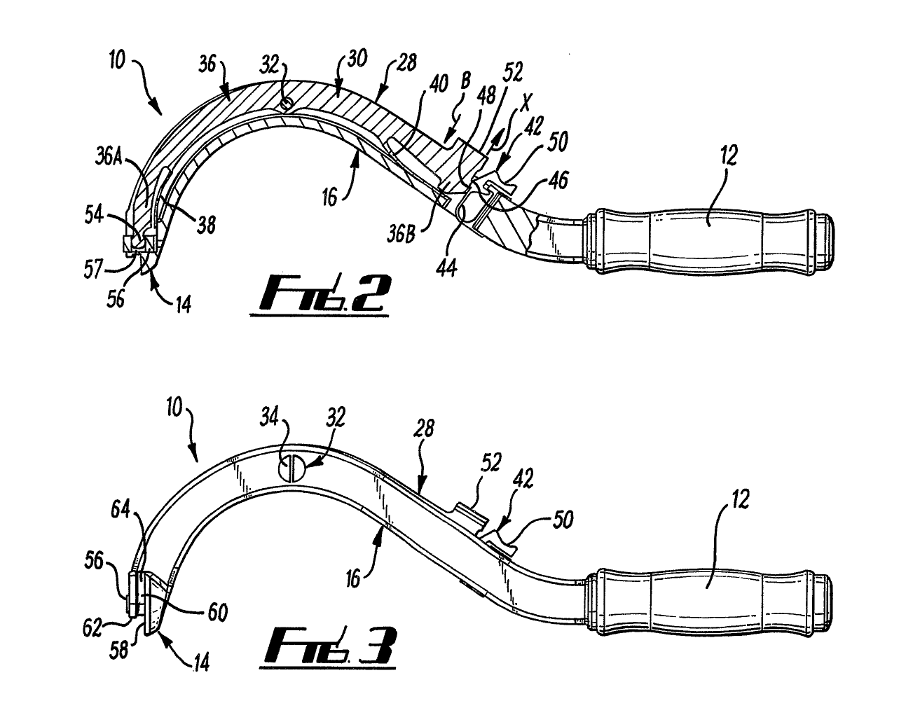

FIG. 3 is a side view of the manipulating device shown in FIGS. 1 and 2;

FIG. 4 is a side view of a holding device of the surgical apparatus;

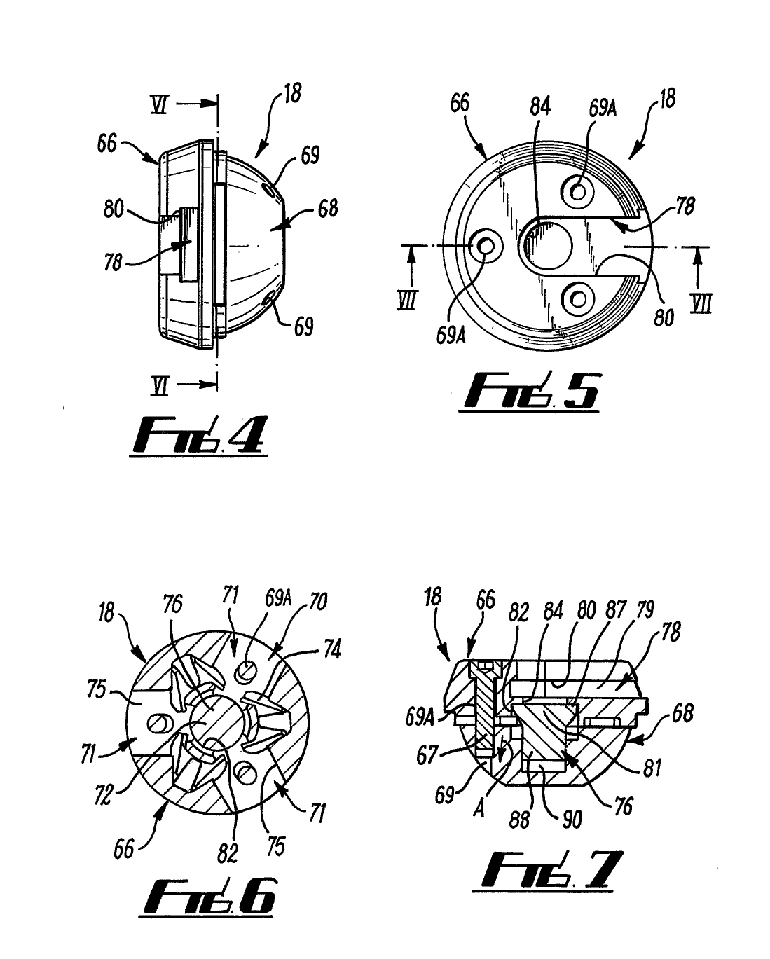

FIG. 5 is a bottom plan view of the holding device shown in FIG. 4;

FIG. 6 is a view along the lines VI-VI in FIG. 4;

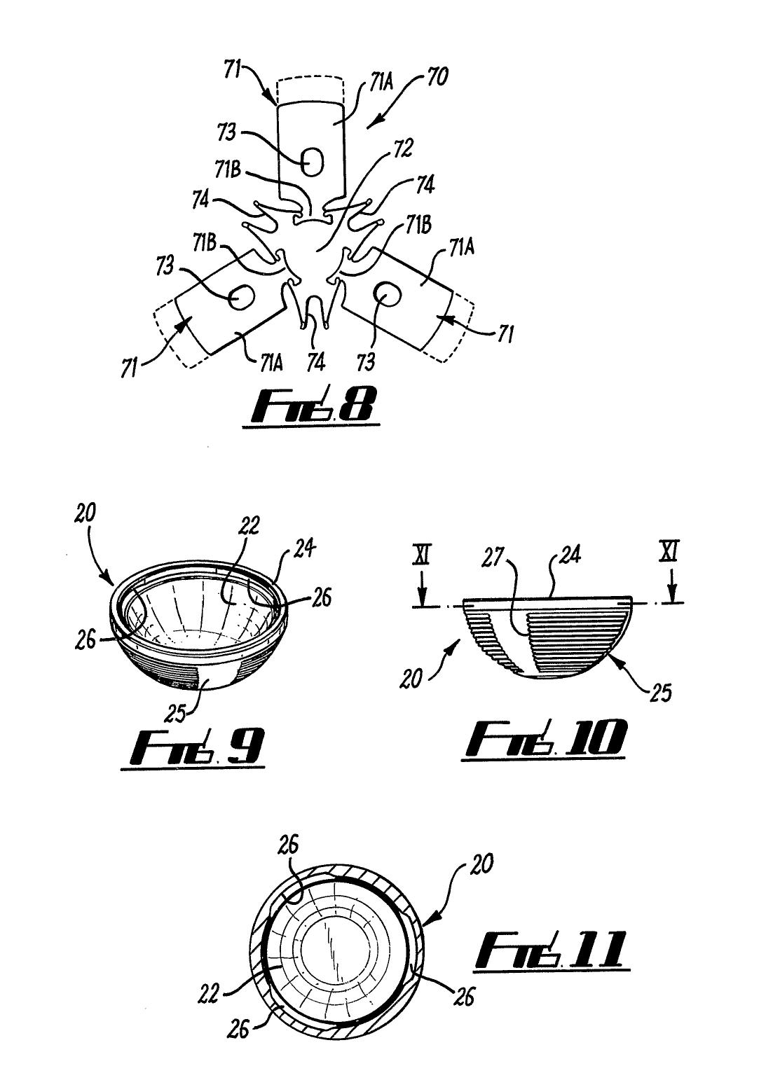

FIG. 7 is a view along the lines VII-VII in FIG. 5;

FIG. 8 is a top plan view of a locking assembly;

FIG. 9 is a perspective view of a surgical member;

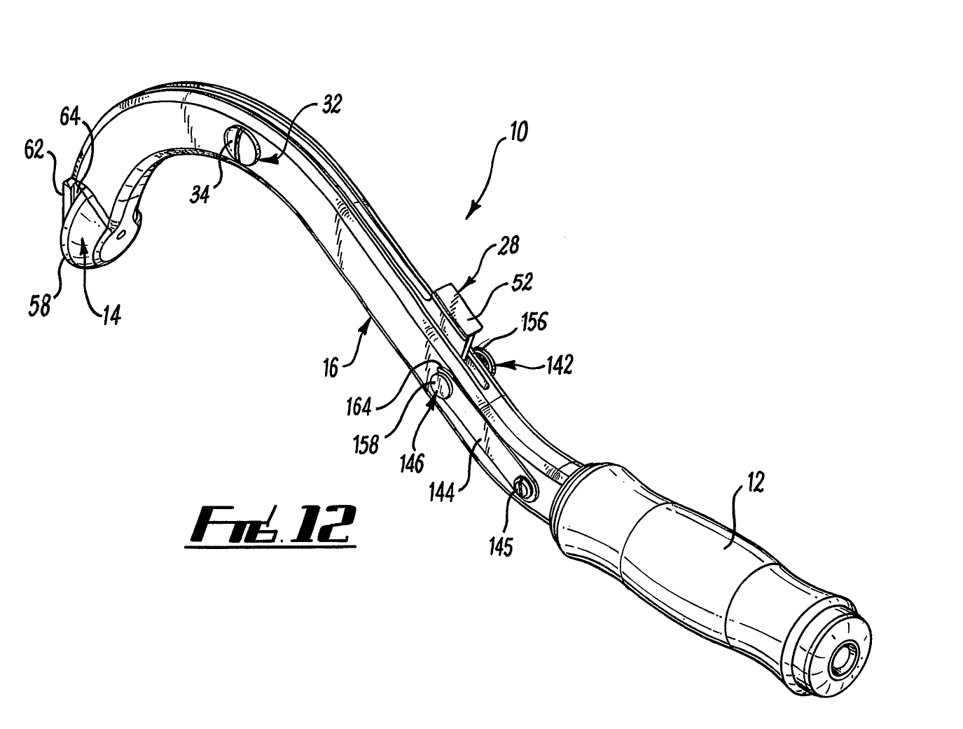

FIG. 10 is a side view of the surgical member shown in FIG. 9;

FIG. 11 is view along the lines XI-XI in FIG. 10;

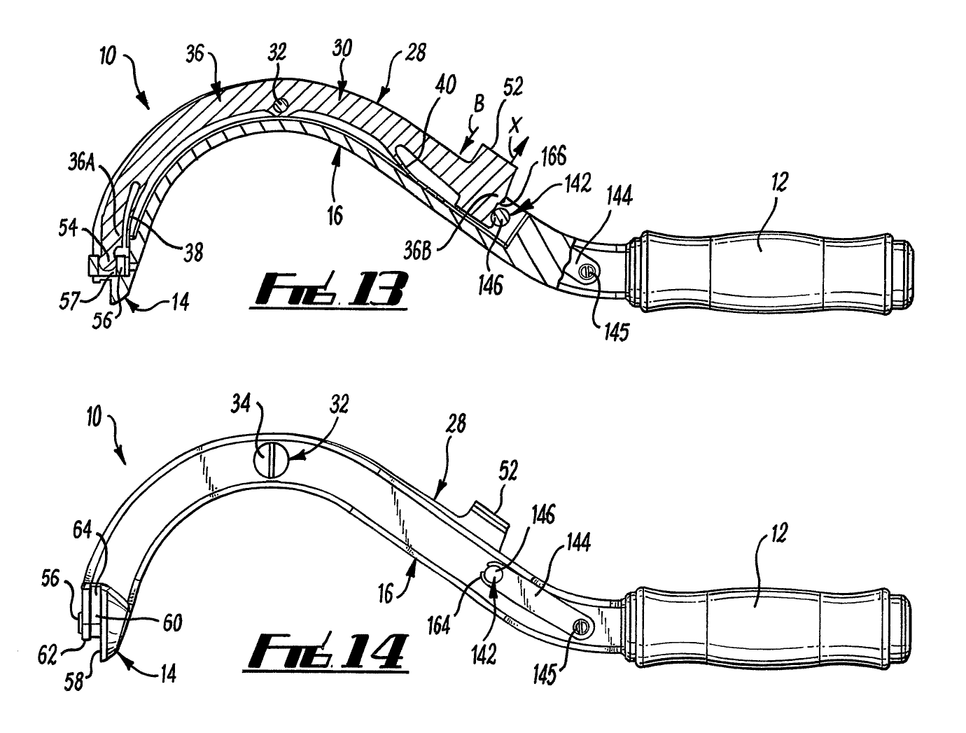

FIG. 12 is a perspective view of a second embodiment of a manipulating device of a surgical apparatus;

FIG. 13 is a part sectional side view of the manipulating device shown in FIG. 12;

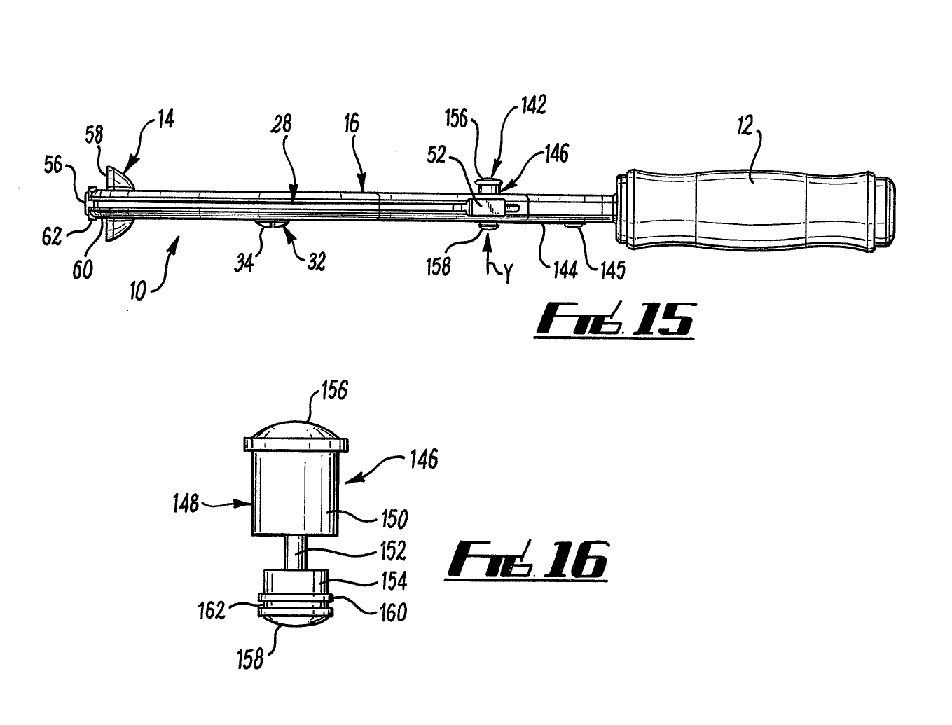

FIG. 14 is a side view of the manipulating device shown in FIG. 12;

FIG. 15 is a top plan view of the manipulating device shown in FIG. 12; and

FIG. 16 is a top plan view of a detent member used in the manipulating device shown in FIG. 12.

The drawings show a surgical apparatus for use in hip replacement surgery, particularly minimal invasive hip replacement surgery

Referring to FIGS. 1 to 3 of the drawings, there is shown a first embodiment of a component of the surgical apparatus. The component comprises a manipulating device 10, which has a manipulating portion in the form of a handle 12, and a delivery portion 14 for delivering a surgical member to the desired position to a patient during surgery. The surgical apparatus shown in FIGS. 1 and 2 is suitable to use in hip replacement surgery and has a particular advantage that it can be used in minimal invasive surgery.

The delivery portion 14 is spaced longitudinally from the handle 12 and is connected to the handle 12 by a curved connecting portion 16. As can be seen from FIG. 2, the connecting portion 16 extends laterally from the delivery portion 14, and curves to handle 12, so that the manipulating device 10 is generally question mark shaped. This provides the advantage, in the embodiment described, that the surgeon can manipulate a cup implant 20 (see FIGS. 9 to 11) to a desired position through an incision formed during the minimal invasive surgery.

The surgical apparatus also includes a holding device 18 (see FIGS. 4 to 8), for holding a surgical member in the form of the cup implant 20 for implantation into the acetabulum of a patient.

The surgical apparatus comprises a securing arrangement for securing the surgical member to the surgical apparatus. The securing arrangement comprises a securing mechanism 28 on the manipulating device 10, and a locking assembly on the holding device 18.

The holding device 18 can be securably mounted on the delivery portion 14 by the securing mechanism 28 on the manipulating device 10. The securing mechanism 28 extends through the connecting portion 16.

Referring to FIGS. 9 to 11, the cup implant 20 defines a socket 22 having a hemi-spherical configuration. A ridge 24 extends around the socket 22, and defines three recesses 26 for receipt of locking members (see below) for locking the implant 20 to the holding device 18.

The external surface 25 of the cup implant 20 is coated with hydroxyapatite to provide a primary fixing to the bone, when the cup implant 20 is implanted into the acetabulum of a patient.

While not wishing to be limited to a particular theory, hydroxyapatite, being a ceramic calcium phosphate material, is a material similar to bone. The bone into which the cup implant 20 is implanted grows to connect with the hydroxyapatite, thus providing a primary fixing of the implant in the acetabulum.

The external surface 25 is provided with a ridged pattern 27 formed of alternating ridges and grooves. The ridged pattern 27 provides a secondary fixing of a mechanical nature, if the hydroxyapatite coating material is either absorbed by the body or destroyed by infection. The bone, which fills the grooves of the pattern 27, acts as a key for the implant.

Referring to FIG. 2, the securing mechanism 28 comprises a lever assembly 30, which is pivotally mounted by a pivot 32 to the connecting portion 16. The pivot 32 is in the form of a screw having a large head 34 to allow for easy dismantling.

The lever assembly 30 comprises a lever 36 having a distal end region 36A adjacent to the delivery portion 14, and a proximal end region 36B adjacent to the handle 12.

The lever 36 is curved to conform to the shape of the connecting portion 16. The lever 36 is pivotally mounted on the pivot 32 and can be pivoted to move the distal end region 36A in a direction shown by the arrow X to a release position, in which the holding device 18 can be arranged on, or removed from, the delivery portion 14. The lever 30 can also be pivoted to move the distal end region 36A in the opposite direction to a locking position, in which the holding device 18 can be locked to the delivery portion 14, and in which the lever assembly 30 can co-operate with the implant 20 to lock the implant 20 to the delivery portion 14.

The lever assembly 30 further includes an urging arrangement, in the form of first and second springs 38, 40 arranged respectively that the distal and proximal end regions 36A, 36B of the lever 36. The first and second springs 38, 40 are in the form of leaf springs.

The first and second springs 38, 40 engage the connecting portion 16, as shown in FIG. 2, and urge the lever assembly 30 to an intermediate position between the release position and the locking position. In the intermediate position, the holding device 18 is locked to the delivery portion 14, but the implant 20 is released from the holding device 18. The lever 36 is movable between the release and the locking positions in a main plane of movement of the lever 36.

A fastener 42 is provided to fasten the lever assembly 30 in the locking position. The fastener 42 is mounted on the connecting portion 16 and comprises a spring member 44 having a detent formation 46 provided thereon. The detent formation 46 co-operates with a corresponding detent element 48 on the proximal end region 36B of the lever 36, when the fastener 42 is in a fastening position.

A release button 50 is provided on the spring member 44 to release the detent formation 46 from the detent element 48 and allow of the lever assembly 30 to move to the intermediate position from the locking position. The release button 50 can be depressed to move the detent formation 46 away from the detent element 48, to a non-fastening position. Thus, as shown in FIGS. 1 to 3, the fastener 42 moves within the main plane of movement of the lever 36.

A locking button 52 is provided on the lever 36 which, when depressed, allows the lever assembly 30 to move to the locking position.

At the distal end region 36A of the lever 36 there is provided an attaching member 54 for releasably attaching to the lever 36 a projection member in the form of a pin 56. The pin 56 defines a hole 57 in which the attaching member 54 is received to mount the pin 56 on the lever 36.

The pin 56 projects from the delivery portion 14 when the lever assembly 30 is in the intermediate position, and the project further from the delivery portion 14 when the lever assembly 30 is in the locking position. The purpose of the pin 56 is described below.

The delivery portion 14 comprises a platform surface 58 and a raised portion 60 extending from the platform surface 58. A flange 62 extends from the raised portion 60 substantially parallel with the platform surface 58. The flange 62 defines, with the platform surface 58, a securing channel 64. The securing channel 64 is provided to co-operate with a corresponding formation on the holding device 18, as described below.

Referring to FIGS. 4 to 8, there is shown the holding device 18, which comprises a support member 66 and a cover member 68. The cover member 68 is formed of an acetal or similar suitable material, and is of a truncated dome shaped configuration. The cover member 68 is provided to protect the internal surface of the socket to 22 from damage. The cover member 68 defines of three threaded through apertures 69 to receive suitable fixing members, such as screws 67 to fix the cover member 68 to the support member 66.

The support member 66 defines corresponding through apertures 69A arranged to align with the through apertures 69. The screws 67 can be received via the through apertures 69A to be threadably received in the through apertures 69in the cover member 68.

A locking assembly 70 is supported on the support member 66 and is held on the support member 66 by the cover 68. The locking assembly 68 comprises three locking members 71 which are circumferentially equispaced from one another around a central space 72.

Each locking member 71 comprises a main part 71A, which is received in the elongate grooves 26 in the implant 20, thereby locking the implant 20 to the holding device 18. Each locking member 71 also comprises a cam engaging part 71B for engaging a cam member 76, also as explained below. Each main part 71A defines an oval aperture 73 through which the screws 67 can to allow the movement of the locking members 71.

Referring to FIG. 8, the locking assembly 70 is shown. Each of the locking members 71 is movable from a release position shown in solid lines in FIG. 8, to a locking position shown in broken lines in FIG. 8. This movement of the locking members 71 is effected by a cam member 76 movably mounted in the support member 66.

A spring arrangement in the form of three springs 74 is provided. Each of the springs 74 is arranged between adjacent locking members 71 and urges the locking members 71 to the release position. Each of the springs 74 is generally M-shaped.

The support member 66 defines a shaped guide recess or socket 75 to receive the locking assembly 70 and to guide the movement of the locking members 71. The guide socket 75 is of generally the same shape as the locking assembly 70.

On the opposite face of the support member 66 to the guide recess 75, there is defined an elongate recess 78. The elongate recess 78 is defined by a wall 79, provided in the support member 66, and an inwardly directed shoulder 80. The elongate recess 78 can co-operate with the securing channel 64 on the delivery portion 14. The shoulder 80 is slidably received in the securing channel 64.

The support member 66 defines a circular recess 82 in which the cam member 76 is arranged. The cam member 76 has a frustoconical portion 78 received in the circular recess 82. The wall of the circular recess 82 provides an inwardly directed shoulder 87 which defines a circular aperture 84 concentric with, but offset axially from, the circular recess 82. The circular recess 82 communicates with the elongate recess 78 via the circular aperture 84.

The circular aperture 84 extends to the elongate recess 78 and receives therein the projecting end of the pin 56, when the holding device 18 is mounted on the delivery portion 14 and the lever assembly 30 is in the intermediate position.

As explained above, when the lever assembly 30 is in the intermediate position, an end region of the pin 56 projects from the delivery portion 14. This has the advantage in the embodiment described, that when the lever assembly 30 is in the intermediate position, and the holding device 18 is mounted on the delivery portion 14, the end region of the pin 56 projecting from the delivery portion 14 is received in the circular aperture 84, thereby preventing removal of the holding device 18 from the delivery portion 14.

The cam member 76 is movable by the action thereon of the pin 56, when the holding device 18 is mounted on the delivery portion 14 and the lever assembly 30 is moved to the locking position. During such movement of the lever assembly 30, the pin 56 engages the cam member 76 to urge it in the direction shown by the arrow A in FIG. 7.

The cam member 76 comprises the frustoconical portion 81 and a generally cylindrical portion 88, which extends from the frustoconical portion 81. The surface of the frustoconical portion 81 engages the cam engaging members 71B, and on further movement of the cam member 76 in the direction of the arrow A, the frustoconical portion 81 urges the locking members 71 radially outwardly to the locking position. The cylindrical portion 88 is received in a correspondingly shaped bore 90 in the cover member 68.

When the lever assembly 30 is moved to the release position, the pin 56 is retracted and the cam 76 moves in the direction opposite to the arrow A. The locking members 71 move radially inwardly to the release position, thereby releasing the cup implant 20 and allowing it to be removed from the holding device 18.

Thus, in use, the locking button 52 is moved the in the direction of the arrow X (see FIG. 2) to retract the pin 56 fully into the delivery portion 14. This allows in the holding device 18 to be slid onto the delivery portion 14 with the shoulder 80 being received in the securing channel 64.

The first holding device 18 is then selected from a plurality of holding devices of different sizes, each different size holding device 18 is designed for a different size of implant 20.

When the holding device 18 is mounted on the delivery portion 14, the lever assembly 30 is allowed to move to the intermediate position, by releasing the locking button 52. The end region of the pin 56 is then received in the aperture 84 to secure the holding device 18 on the delivery portion 14.

The appropriate implant 20 is then mounted on the holding device 18, with the elongate grooves 26 aligned with the locking members 71. When the user depresses the locking button 52, the lever assembly 30 moves the pin 56 further outwardly of the delivery portion 14. The detent formation 46 of the fastener 42 engages the detent formation 48 on the lever 36 to lock the lever assembly 30 in the locking position.

The movement of the pin 56, described in the preceding paragraph, brings it into engagement with the cam member 76, and the pin 56 pushes the cam member 76 in the direction of the arrow A in FIG. 7, thereby urging the locking members 71 radially outwardly, that such that the end regions of the locking members 71 are received in the elongate grooves 26 of the cup implant 20, thereby locking the implant 20 on the holding device 18

The implant 20 can then be manipulated by the use of the surgical apparatus into position during surgery and placed in the patient's acetabulum. The implant 20 can be released from the holding device 18 by pressing the release button 50 of the fastener 42, which releases the lever assembly 30 to allow the lever assembly 30 to return to the intermediate position by the action of the first and second springs 38, 40.

When the lever assembly 30 returns to the intermediate position, the pin 56 moves away from the cam member 76, thereby causing the cam member 76 to move in the opposite direction to the arrow A by the force applied thereto by the locking members 71 being urged inwardly by the springs 74.

The inward movement of the locking members 71 releases the implant 20 from the holding device 18 thereby allowing the implantation to be completed. The construction of the above described embodiment has the advantage that the surgeon can release the cup implant 20 without disturbing its position in the acetabulum.

Referring to FIGS. 12 to 16, there is shown a further embodiment of the manipulating device 10, which comprises many of the same features as shown in FIGS. 1 to 3, and these features have been given the same reference numerals as in FIGS. 1 to 3.

The embodiment shown in FIGS. 12 to 16 differs from the embodiment shown in FIGS. 1 to 3 in that the fastener 42 is omitted and, instead, a fastener 142 is employed.

The fastener 142 employed in the embodiment shown in FIGS. 12 to 16 differs from the fastener 42 shown in FIGS. 1 to 3 in that the fastener 142 moves at right angles to the main plane of movement of the lever 36.

The fastener 142 comprises a spring member in the form of a leaf spring 144. The leaf spring 144 is mounted externally on the connecting portion 16 and secured thereto by screw 145 at one end of the leaf spring 144.

The leaf spring 144 is attached at its opposite end to a detent member in the form of a pin 146, which extends through the connecting portion 16 at right angles to the main plane of movement of the lever 36. The spring 144 acts to urge the pin 146 in the direction of the arrow Y in FIG. 15.

The pin 146 comprises a generally cylindrical main body 148 having a first end region 150 of a wide diameter, a central region of a narrow diameter 152, and a second end region 154 of an intermediate diameter. The first and second end regions 150, 154 and the central region 152 are generally coaxial with each other.

At each end of the main body 148, there is provided respective first and second domed formations 156, 158 to allow the pin 146 to be pushed into the connecting portion 16 from either side of the connecting portion 16.

The first domed formation 156 is provided on the first end region 150 of the main body 144. The second domed formation 158 is provided on the second end region 154 of the main body 158. Each of the first and second domed formations 156, 158 is wider than the end of the main body 148 on which it is provided. The second end region 154 of the main body 148 is provided with a flange 160 spaced from the second domed formation 158.

The spring 144 has a C-shaped end 164 to engage around the second end region 154 of the main body 148 in the gap 162 between the second domed formation 158 and the flange 160. The C-shaped end of the spring 144 attaches the spring 144 to the pin 146 and the flange 160 enables the spring 144 to push the pin 146 in the direction of the arrow Y.

The proximal end of the lever 36 has a detent recess 166 therein which can receive the second end region 154 of the pin 146. When the locking button 52 is depressed in the direction of the arrow B to move the lever 30 to its locking position, the detent recess 166 receives the second end region 154 of the pin 144, thereby securing the lever 36 in its locking position.

The pin 146 can be pushed in the opposite direction to the arrow Y until the first domed formation 156 engages the side of the connecting portion 16. In this position, the central region 152 of the pin 146 is aligned with the proximal end region of the lever 36, thereby allowing the lever 36 to move to its intermediate position.

When the locking button 52 is depressed in the direction of the arrow B to move the lever 36 to the locking position, the detent recess 166 is moved into alignment with the second end region 154 of the pin 146. In this position, the spring 144 moves the pin 146 in the direction of the arrow Y so that the second end region 154 is received within the detent recess 166, thereby securing the lever 36 in the locking position.

When it is desired to release the lever 36 from the locking position, for example to release an implant, the first domed formation 156 is pressed. This moves the pin 146 in the direction opposite to the arrow Y until the central region 152 of the pin 146 is aligned with the proximal end regions 36B of the lever 36. the alignment of the central region 152 with the proximal end region 36B allows the lever 36 to be moved to its release position to release the implant.

There is thus described a surgical apparatus which can be used in minimal invasive hip replacement surgery, and which allows the surgeon to manipulate a replacement cup implant to the correct position during such surgery.

The manipulating device 10, the support member 18 and the locking assembly 70 may be formed of a suitable strong material, such as stainless steel. An example of such a material is sold by the company Carpenter under the name Custom 455 stainless steel.

Various modifications can be made without departing from the scope of the invention, for example, the precise shape of the apparatus, particularly the manipulating device 10 could differ from that shown in the drawings.

1. A surgical apparatus comprising a manipulating portion, a delivery portion spaced longitudinally from the manipulating portion, and a connecting portion connecting the manipulating portion to the delivery portion, wherein the connecting portion extends laterally from the delivery portion, and the surgical apparatus further includes a securing arrangement for releasably securing a surgical member to the delivery portion.

2-70. (canceled)

71. A surgical apparatus according to claim 1, wherein the surgical apparatus further includes a holding device on which the surgical member is mounted, the holding device being releasably securably mountable on the delivery portion, and the holding device comprising a locking assembly for releasably locking the surgical member to the holding device, and wherein the holding device comprises a support member configured to support the surgical member, the support member having substantially the same plan profile as the surgical member.

72. A surgical apparatus according to claim 71, wherein the locking assembly is provided on the support member, the locking assembly being movable between a locked position to lock the surgical member to the holding device, and an unlocked position to allow the surgical member to be mounted on, or removed from, the holding device, the locking assembly comprising a plurality of locking members, wherein each locking member extends radially outwardly from a central region, and each locking member is spaced circumferentially from the other locking members.

73. A surgical apparatus according to claim 72, wherein a spring arrangement is provided to urge each locking member to the unlocked position, the locking members being circumferentially arranged.

74. A surgical apparatus according to claim 73, wherein the spring arrangement comprises a plurality of spring elements arranged between adjacent locking members, and each spring element is of an M shaped configuration, and is connected at its opposite ends to adjacent locking members.

75. A surgical apparatus according to claim 71, wherein the support member defines a socket to receive the locking assembly, the socket being configured to be generally the same shape as the locking assembly, and to guide the locking assembly between the locked and the unlocked positions.

76. A surgical apparatus according to claim 71, wherein the delivery portion comprises a platform surface on which the holding device can be mounted, and a securing formation is provided on the platform surface, the securing formation comprising a raised portion extending from the platform surface, and wherein a flange member extends from the raised portion, the flange member extending generally parallel to the platform surface, and defining with the platform surface, a securing channel to co-operate with a corresponding formation on the holding device.

77. A surgical apparatus according to claim 72, wherein the delivery portion comprises a projection member operable to move the locking assembly to the locked position, and the holding device further including a cam member for translating movement from the projection member to each locking member, and wherein each locking member comprises a main portion for engaging the surgical member, and a cam engaging portion for engaging the cam member, the projection member engaging the cam member to move the cam member away from the delivery portion, whereby the cam surface engages each locking member to move each, locking member outwardly.

78. A surgical apparatus according to claim 77, wherein the cam member is of a generally conical configuration, having a conical, or frustoconical, cam surface, and further including a generally cylindrical region, the cam member engaging the, or each, locking member to move the, or each, locking member to the locked position, and the holding device comprising a cover member to hold the locking assembly on the support member, the cover member defining a bore or aperture to guide the cam member, and the generally cylindrical region being received in the bore or aperture in the cover member.

79. A surgical apparatus according to claim 71, wherein the holding device is slidably mounted on the delivery portion, and the support member has a wall and an inwardly directed shoulder configured to co-operate with the securing formation on the delivery portion, the wall and the shoulder defining an elongate recess to receive the securing formation on the delivery portion.

80. A surgical apparatus according to claim 1, wherein the securing mechanism comprises a lever assembly having a proximal end at, or adjacent to, the manipulating portion, and a distal end at, or adjacent to, the delivery portion, and the lever assembly being pivotally mounted on the connecting portion, the lever assembly being movable between a release position, in which the holding device can be arranged on or removed from the delivery portion, and a locking position, in which the holding device can be locked to the delivery portion, and in which locking position, the lever assembly can co-operate with the securing mechanism to secure the surgical member to the holding device.

81. A surgical apparatus according to claim 80, wherein the lever assembly is movable between the release and the locking positions in a main plane of movement of the lever assembly, and the lever assembly is movable to an intermediate position, in which the fastening mechanism can release the surgical member from the holding device, and in which intermediate position, the holding device can be locked to the delivery portion, the intermediate position being between the release position and the locking position.

82. A surgical apparatus according to claim 81, wherein the lever assembly comprises an urging arrangement to urge the lever assembly to the intermediate position from the release position, and to urge the lever assembly to the intermediate position from the locking position.

83. A surgical apparatus according to claim 82, wherein the urging arrangement comprises first and second springs, each spring being arranged at a respective opposite end region of the lever,

84. A surgical apparatus according to claim 83, wherein one of the first and second springs is arranged to urge the lever assembly from the release position to the intermediate position, and the other of the first and second springs is arranged to urge the lever assembly from the locking position to the intermediate position, and each of the first and second springs extends from the lever to engage the connecting portion, and each leaf spring has a first end attached to the lever, and a second end in contact with the connecting portion.

85. A surgical apparatus according to claim 84, wherein both of the first and second springs are arranged to urge the lever assembly from the release position to the intermediate position, and both of the first and second springs are arranged to urge the lever assembly from the locking position to the intermediate position, and each of the first and second springs extends from the lever to engage the connecting portion, and each leaf spring has a first end attached to the lever, and a second end in contact with the connecting portion.

86. A surgical apparatus according to claim 80, wherein a pivot member is provided in the connecting portion, and the lever assembly is pivotally mounted on the pivot member to allow pivoting of the lever assembly about the pivot member.

87. A surgical apparatus according to claim 80, wherein the lever assembly extends to the delivery portion and the projection member is provided on the lever assembly to engage the holding device, thereby locking the holding device to the delivery portion when the lever assembly is in the locking or the intermediate position.

88. A surgical apparatus according to claim 80, wherein when the lever assembly is in the intermediate position, the projection member projects from the delivery portion, and the projection member can be received by the holding device to secure the holding device on the delivery portion when the lever assembly is in the intermediate position.

89. A surgical apparatus according to claim 80, wherein the lever assembly includes a fastener to fasten the lever assembly in the locking position, the fastener comprising a detent member, which is co-operable with a corresponding detent element on the lever assembly, the fastener being movable between a fastening position, in which the lever assembly is fastened in the locking position, and a non-fastening position, in which the lever assembly can be moved from the locking position to the intermediate position.

90. A surgical apparatus according to claim 89, wherein the fastener includes an urging element to urge the fastener to the fastening position, the fastener being movable between the fastening and the non-fastening positions substantially in the main plane of movement of the lever assembly, and the urging element is held within the connecting portion.

91. A surgical apparatus according to claim 90, wherein the fastener comprises a pin movable between the fastening and the non-fastening positions transverse to the main plane of movement of the lever assembly, the pin being movable between the fastening and the non-fastening positions at right angles to the main plane of movement of the lever assembly, and wherein the pin comprises an engagement end region configured to engage the lever assembly, the lever assembly defines a detent recess to receive said engagement end region to hold the lever assembly in the locking position, the pin includes a narrow region adjacent the aforesaid end region, and the lever assembly can move between its positions when the narrow region is aligned with the lever assembly.
